(19) United States
(12) Patent Application Publication
(10) Pub. No.: US 2009/0053319 A1
(43) Pub. Date: Feb. 26, 2009

(54) Sore Throat Relief Composition And Method of Producing Same

(76) Inventor: Wayne Jeffrey Perry, Albany, NY (US)

Correspondence Address: AMIN HALLIHAN, LLC, 444 NORTH ORLEANS STREET, SUITE 400, CHICAGO, IL, 60654, US

(21) Appl. No.: 11/842,458

(22) Filed: Aug. 21, 2007

(51) Int. Cl. A61K 31/165; A61K 35/64; A61P 11/00

(52) U.S. Cl. 424/537; 514/625; 514/627

(57) ABSTRACT

The present invention provides a sore throat relief composition and a method of producing the composition comprising oleoresin capsicum containing capsaicin, dihydrocapsaicin, nordihydrocapsaicin, homodihydrocapsaicin, and homocapsaicin as active ingredients, combined with vegetable glycerin, purified water, spearmint oil, wild cherry bark, clove honey, and ascorbic acid for fully relieving and preventing chronic and occasional sore throat symptoms, including pain, dryness, and inflammation.

### FIELD OF THE INVENTION

The present invention relates to a sore throat relief composition containing natural capsaicinoids for relieving sore throat pain, dryness, and inflammation along with providing antimicrobial properties. The present invention is related to a copending application, U.S. Ser. No. 11/731,656, entitled SINUS RELIEF COMPOSITION AND METHOD OF PRODUCING THE SAME filed by the inventor with the United States Patent Office and which disclosure is incorporated herein.

### BACKGROUND OF THE INVENTION

Capsaicinoids are the active components in chili peppers and plants belonging to the Capsicum family. The pungency level of a plant belonging to the Capsicum family is measured on the Scoville scale. The greater the number of Scoville heat units indicates the “hotter” the plant. Capsaicin is a capsaicinoid and the most prevalent capsaicinoid in chili peppers, followed by dihydrocapsaicin, nordihydrocapsaicin, homodihydrocapsaicin, and homocapsaicin. Pure capsaicin ranges between 15,000,000-16,000,000 Scoville units. Dihydrocapsaicin is an irritant and has a similar pungency to capsaicin. Nordihydrocapsaicin, homodihydrocapsaicin, and homocapsaicin are also irritants and have a pungency of about 8,600,000-9,100,000 Scoville units.

Each capsaicinoid and its corresponding chemical structure is shown below.

| | |
|---|---|
| Capsaicin | 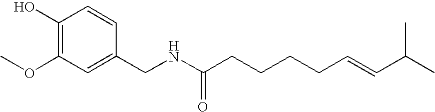 |
| Dihydrocapsaicin | 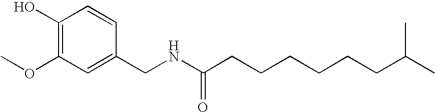 |
| Nordihydrocapsaicin | 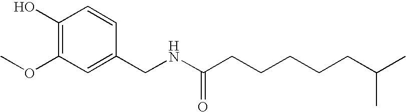 |
| Homodihydrocapsaicin | 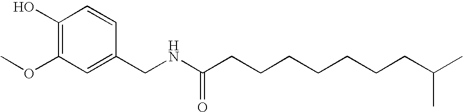 |
| Homocapsaicin | 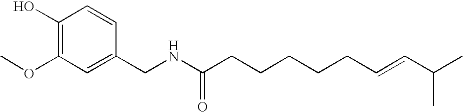 |

Capsaicinoids are irritants and produce a sensation of burning or hotness when they come in contact with human tissue. Capsaicinoids chemically interact with sensory neurons by binding to thermoreceptor nerve endings inside the body, or receptors in the skin. These receptors can be stimulated with heat or physical abrasion causing chemical signals to pass through the cell membrane and into the cell which causes the neuron to generate its own signal to the brain. The capsaicinoid molecules induce the same effect of producing a burning sensation, but not a chemical burn, that heat or physical abrasion does by binding to the thermoreceptor nerve endings. Therefore, capsaicinoids stimulate the nerve endings which leads to desensitization, a process that strategically provides an analgesic effect that suppresses pain and inflammation more quickly and for a longer period of time than other analgesics.

Capsaicinoids have many uses such as a heat additive for foods, topical ointments, and non-lethal weapons. Capsaicinoids are used in foods to add spice or “heat”, such as in hot sauce or salsa. Capsaicinoids are also used as a topical ointment to relieve minor aches and pains in muscles and joints, such as symptoms associated with arthritis. Capsaicinoids are also used as an ingredient in the non-lethal weapon commonly known as “pepper spray”, which when sprayed into the eyes or onto the skin is painful to the recipient.

Current treatments for relief of sore throat pain, dryness and inflammation include a variety of analgesics, antibiotics such as penicillin, pain relievers such as ibuprofen or acetaminophen, throat lozenges, prescription and over the counter drug allergy medicines, gargling salt water, hydrating, and/or using a steam vaporizer. However, these current treatments are mostly unnatural and may require a prescription for utilization.

### SUMMARY OF THE INVENTION

One object of the present invention is for a natural, effective composition for the relief of general sore throat symptoms.

Another object of the present invention is a composition having components which provide a synergistic effect to kill germs and bacteria that cause sore throat pain and symptoms.

Still another object of the present invention is for a composition which provides relief of and prevents bacterial and non-bacterial related sore throat symptoms.

A further object of the present invention is a composition for the relief of chronic and occasional sore throat symptoms having the beneficial effects of capsaicinoids without the lingering pain and discomfort of pure pepper.

In accordance with the present invention, a natural composition containing capsaicinoids is used for fully relieving and preventing sore throat symptoms, including pain, dryness, and inflammation has been developed. In one form of the present invention, the composition has been developed to be administered orally.

The sore throat relief composition in accordance with the invention includes oleoresin capsicum including capsaicin, dihydrocapsaicin, nordihydrocapsaicin, homodihydrocapsaicin, and homocapsaicin as active ingredients that has been developed specifically for the purpose of relieving chronic and occasional sore throat symptoms. Additionally, the sore throat relief composition relieves symptoms associated with colds, flu, throat infections, bacterial infections, post nasal drip and strained throat muscles.

In one embodiment of the present invention, the sore throat relief composition includes 0.25 mL to 5 mL oleoresin capsicum per 5 gallons of purified water or liquid suspension material.

In another embodiment of the present invention, the sore throat relief composition includes oleoresin capsicum including capsaicin, dihydrocapsaicin, nordihydrocapsaicin, homodihydrocapsaicin, and homocapsaicin as active ingredients and having a heat range of 100,000 to 500,000 Scoville units.

In yet another embodiment of the present invention, the sore throat relief composition includes oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin.

In still another embodiment of the present invention, the oleoresin capsicum may be water soluble.

Additionally, the sore throat relief composition can also include spearmint oil, wild cherry bark, clove honey, vegetable glycerin, purified water, and ascorbic acid.

The present invention relates also to a method of producing the sore throat relief composition. The method includes storing the oleoresin capsicum and purified water between 60 and 65 degrees Fahrenheit; adding the vegetable glycerin and clove honey to the purified water; agitating the solution to full solubility; adding the ascorbic acid and agitating the solution to full solubility; adding the wild cherry bark and spearmint oil and agitating the solution to full solubility; and adding the oleoresin capsicum and agitating the solution for at least two minutes and allowing the oleoresin capsicum to reach full solubility without the loss of effectiveness.

These and other embodiments of the present invention are more fully described in connection with the detailed description.

### DETAILED DESCRIPTION OF THE INVENTION

The present invention relates to a natural composition containing capsaicinoids used for fully relieving and preventing chronic and occasional sore throat symptoms, including pain, dryness, inflammation, and infection. The present composition is designed to be administered orally.

One embodiment of the present invention relates to a sore throat relief composition having oleoresin capsicum including capsaicin, dihydrocapsaicin, nordihydrocapsaicin, homodihydrocapsaicin, and homocapsaicin as active ingredients, combined with spearmint oil, wild cherry bark, clove honey, vegetable glycerin, ascorbic acid, and purified water.

The synergistic effect of the components of the sore throat relief composition of one form of the invention relieve chronic and occasional sore throat symptoms without the lingering pain and discomfort of pure pepper. The sore throat relief composition of one embodiment of the present invention also relieves symptoms associated with colds, flu, throat infections, bacterial infections, post nasal drip and strained throat muscles.

Oleoresin capsicum, a natural resin, is a concentrated extract of capsaicinoids from several Capsicum varieties. The Capsicum variety utilized in the present invention is derived from cayenne pepper plants, which includes Capsicum baccatum and Capsicum frutescens. The oleoresin capsicum includes capsaicin, dihydrocapsaicin, nordihydrocapsaicin, homodihydrocapsaicin, and homocapsaicin as active ingredients and the main heat component of Capsicum variety peppers. Preferably, the oleoresin capsicum utilized in the present invention is all natural, water soluble or soluble in a liquid suspension material. The use of natural oleoresin capsicum including capsaicin, dihydrocapsaicin, nordihydrocapsaicin, homodihydrocapsaicin, and homocapsaicin as opposed to synthetic capsaicin allows for repeated use and effective delivery without too much heat. However, it is within the scope of the present invention that a combination of synthetic capsaicinoids including about 67-71% capsaicin, about 20-24% dihydrocapsaicin, about 5-9% nordihydrocapsaicin, about 0.25-2% homodihydrocapsaicin, and about 0.25-2% homocapsaicin could also be used.

The capsaicinoids in the oleoresin capsicum deplete the chemicals which cause pain and swelling, such as Substance P which is responsible for transmitting pain in the nervous system. The depletion of these chemicals relieves sore throat pain and the associated inflammation and dryness.

The oleoresin capsicum relieves sore throat symptoms by flushing out, for example, bacteria, excess mucous, and allergens including dust mites, mold, fungi and pollen. The oleoresin capsicum stimulates the nerve endings in the lining of the throat thereby causing the nerve endings to swell to facilitate release of the toxins, bacteria, excess mucous, and allergens, which relieves the sore throat symptoms. The oleoresin capsicum also provides antiseptic properties and kills bacteria which are associated with throat infections.

The spearmint oil clears chest congestion and alleviates post nasal drip, which relieves sore throat symptoms. The spearmint oil relieves dryness and helps to retain moisture in the throat. Finally, the spearmint oil has a pleasant taste to give the user a fresh sensation throughout the throat, mouth and nasal cavities.

The wild cherry bark is a natural cough suppressant which also relieves sore throat dryness and itchiness on the lining of the throat.

The clove honey is a natural preservative which protects and stabilizes the sore throat relief composition. The clove honey also provides antiseptic properties which kill bacteria in the throat. Finally, the clove honey also coats the lining of the throat to increase the amount of time of the pain relief from the sore throat relief composition.

The vegetable glycerin is a natural preservative which protects and stabilizes the sore throat relief composition. The vegetable glycerin also assists the capsaicinoids contained in the oleoresin capsicum to maintain their potency and effectiveness for extended periods of time. The vegetable glycerin maintains maximum effectiveness of the sore throat relief composition by drawing moisture to the lining of the throat which assists in relieving dryness. Additionally, the vegetable glycerin shortens the length of time of any burning sensation associated with the capsaicinoids in the oleoresin capsicum without reducing its effectiveness. The vegetable glycerin stabilizes the formulation, lengthens the shelf life, does not promote the formation of yeast in the composition, shortens the length of time of the burning sensation associated with the capsaicinoids and preserves the potency of the capsaicinoids for up to five times longer than other natural preservatives, such as aloe vera.

The ascorbic acid naturally preserves the sore throat relief composition by adjusting the pH levels. Additionally, the ascorbic acid (vitamin C) supports the immune system and helps to prevent and flush out bacteria growth that causes throat infections.

Finally, the purified water dilutes the sore throat relief composition and permits the sore throat relief composition to secrete directly into the human body's natural water in the throat, mouth and lining of the throat.

In another embodiment of the present invention, the sore throat relief composition may be administered or dispensed through an oral spray wherein the sore throat relief composition is released in a metered dosage. This administration route instantly depletes pain by permitting the composition to directly enter the throat. The sore throat relief composition is sprayed directly into the mouth and toward the throat. The sore throat relief composition may be administered with one to three sprays. Further, the sore throat relief composition can be used as needed without drug interactions or other side effects.

In another embodiment of the present invention, the sore throat relief composition may be administered through an oral gargle rinse. The sore throat relief composition may be used as needed by gargling about 0.5 fluid ounces to about 5 fluid ounces per administration. The sore throat relief composition may be gargled for about 30 seconds to about 60 seconds per administration.

In another embodiment of the present invention, the sore throat relief composition may have a flavoring component to provide flavor, such as bubble gum, sour apple, cherry, cotton candy, and/or strawberry flavors.

In accordance with the present invention, the sore throat relief composition includes about 0.25 mL to about 5 mL oleoresin capsicum per 5 gallons of purified water base or liquid suspension material. The sore throat relief composition may include about 0.0010% to about 2.0% oleoresin capsicum per the total water weight of the base. The sore throat relief composition may include oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin as active ingredients. Also, the sore throat relief composition may include oleoresin capsicum including capsaicin, dihydrocapsaicin, nordihydrocapsaicin, homodihydrocapsaicin, and homocapsaicin as active ingredients and possessing a heat range of 100,000 to 500,000 Scoville units. The oleoresin capsicum including capsaicin, dihydrocapsaicin, nordihydrocapsaicin, homodihydrocapsaicin, and homocapsaicin is present in a non-caustic and safe amount.

In other embodiments of the present invention, the oleoresin capsicum may be water soluble,

the spearmint may be an essential oil,

the wild cherry bark may be a tincture,

the vegetable glycerin may be derived from coconuts and palms,

the honey may be derived from clovers

the ascorbic acid may be synthetic, and

the purified water may be steam distilled to closely match the body's natural water makeup.

The following example of one embodiment of the present invention relates to a sore throat relief composition having oleoresin capsicum in 5 gallons of purified water or liquid suspension material, and demonstrates the scope of the present invention. The sore throat relief composition comprises:

about 3.75 mL oleoresin capsicum;

about 9.6 mL spearmint oil;

about 4.8 mL wild cherry bark;

about 700 mL vegetable glycerin;

about 192 ml clove honey; and

about 160 mL ascorbic acid.

Another example of the present invention relates to a sore throat relief composition in 5 gallons of purified water or liquid suspension material, including:

about 0.0010% to about 2.0% by weight of the total water weight of oleoresin capsicum;

about 0.050% to about 0.090% by weight of the total water weight of spearmint oil;

about 0.25% to about 3.0% by weight of the total water weight of clove honey;

about 0.025% to about 0.065% by weight of the total water weight of wild cherry bark;

about 1.0% to about 4.0% by weight of the total water weight of vegetable glycerin; and

about 0.83% by weight of the total water weight ascorbic acid.

One embodiment of the present invention is a sore throat relief kit having a sore throat relief composition having oleoresin capsicum including capsaicin, dihydrocapsaicin, nordihydrocapsaicin, homodihydrocapsaicin, and homocapsaicin as active ingredients, combined with vegetable glycerin, clove honey, purified water, spearmint oil, wild cherry bark tincture, and ascorbic acid; and an instruction for a human to administer the sore throat relief composition orally. One form of the sore throat relief kit includes the sore throat relief composition described above and an instruction for a human to administer the sore throat relief composition orally.

The present invention also includes a method of producing the sore throat relief composition. One embodiment of the method of producing the sore throat relief composition includes storing the oleoresin capsicum and purified water between 60 and 65 degrees Fahrenheit, which increases the water solubility of the oleoresin capsicum. The method further includes adding the vegetable glycerin and clove honey to the purified water first; agitating the first solution to maximize the solubility; adding the ascorbic acid to the first solution to make a second solution and agitating the second solution to maximum solubility; adding the wild cherry bark and spearmint oil to the second solution to make a third solution and agitating the third solution to maximum solubility; and adding the oleoresin capsicum to the third solution and making a fourth solution and agitating the fourth solution and allowing the oleoresin capsicum to reach maximum solubility without losing effectiveness.

In still another embodiment of the present invention, agitating the first solution to maximize the oleoresin capsicum solubility may include agitating the first solution two times wherein each agitation lasts one minute. In another embodiment, agitating the fourth solution permits the oleoresin capsicum to reach maximum solubility without losing effectiveness may include agitating the fourth solution for at least two minutes.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

1. A sore throat relief composition, comprising:
a liquid suspension material,
wild cherry bark, and
oleoresin capsicum including capsaicin, dihydrocapsaicin, nordihydrocapsaicin, homodihydrocapsaicin, and homocapsaicin as active ingredients.
wherein the water soluble oleoresin capsicum is in a concentration of between about 0.0010% to about 2.0% of a total weight of the liquid suspension material.

2. The composition in accordance with claim 1, further comprising:
vegetable glycerin;
spearmint oil;
purified water as the liquid suspension material;
ascorbic acid; and
clove honey.

3. The composition in accordance with claim 2, further comprising a flavoring component.

4. The composition in accordance with claim 1, wherein the composition is administered orally.

5. The composition in accordance with claim 1, wherein the oleoresin capsicum is in a concentration of between about 0.25 mL to about 5 mL per 5 gallons of the liquid suspension material.

6. The composition in accordance with claim 2, wherein the oleoresin capsicum is comprised of about 67-71% capsaicin, about 20-24% dihydrocapsaicin, about 5-9% nordihydrocapsaicin, about 0.25-2% homodihydrocapsaicin, and about 0.25-2% homocapsaicin.

7. (canceled)

8. The composition in accordance with claim 1, wherein the oleoresin capsicum has a heat level of between about 100,000 to 500,000 Scoville units.

9. The composition in accordance with claim 1, wherein the liquid suspension material is purified water and the oleoresin capsicum is water soluble.

10. The composition in accordance with claim 2, wherein the spearmint oil is an essential oil.

11. The composition in accordance with claim 1, wherein the wild cherry bark is a tincture.

12. The composition in accordance with claim 2, wherein the vegetable glycerin is derived from coconuts and palms.

13. The composition in accordance with claim 2, wherein the ascorbic acid is synthetic.

14. A sore throat relief composition containing purified water, comprising:
about 0.0010% to about 2.0% by weight of the total water weight of oleoresin capsicum;
about 0.050% to about 0.090% by weight of the total water weight of spearmint oil;
about 0.025% to about 0.065% by weight of the total water weight of wild cherry;
about 1.0% to about 4.0% by weight of the total water weight of vegetable glycerin;
about 0.25% to about 3.0% by weight of the total water weight of clove honey; and
about 0.83% by weight of the total water weight of ascorbic acid.

15. The composition of claim 14 wherein the oleoresin capsicum is about 0.0195% by weight of the total water weight.

16. A method of producing a sore throat relief composition comprising the steps of:
adding vegetable glycerin and clove honey to purified water to make a first solution;
agitating the first solution;
adding ascorbic acid to the first solution to make a second solution;
agitating the second solution;
adding wild cherry and spearmint oil to the second solution to make a third solution;
agitating the third solution;
adding oleoresin capsicum to the third solution to make a fourth solution; and
agitating the fourth solution.

17. The method in accordance with claim 16, wherein the oleoresin capsicum and purified water are stored between 60 to 65 degrees Fahrenheit.

18. The method in accordance with claim 16, wherein agitating the first solution is done two times for one minute each time.

19. The method in accordance with claim 16, wherein agitating the fourth solution is done for at least two minutes.

20. A method of providing sore throat relief comprising:
administering orally the sore throat relief composition of claim 1.

21. A sore throat relief kit comprising:
the sore throat relief composition of claim 1; and
an instruction for a human to administer the sore throat relief composition orally.
